(19) United States
(12) Patent Application Publication
(10) Pub. No.: US 2024/0083793 A1
(43) Pub. Date: Mar. 14, 2024

(54) METHOD FOR RECOVERY OF BIOLOGICAL PHOSPHORUS FROM AN ACTIVATED SLUDGE WASTEWATER TREATMENT PROCESS

(71) Applicant: DENTRO-P, LLC, Yakima, MA (US)

(72) Inventors: Thomas E. Coleman, Yakima, WA (US); Mari-Karoliina H. Winkler, Seattle, WA (US)

(21) Appl. No.: 18/244,898

(22) Filed: Sep. 11, 2023

Related U.S. Application Data
(60) Provisional application No. 63/405,469, filed on Sep. 11, 2022.

(51) Int. Cl. C02F 3/30; C02F 3/00; C05B 15/00; C12N 1/20

(52) U.S. Cl. CPC C02F 3/308; C02F 3/006; C05B 15/00; C12N 1/20; C02F 2001/007

(57) ABSTRACT

A method for processing of municipal and industrial wastewater in waste activated sludge treatment (WAS) as configured to achieve enhanced biological phosphorus removal (EBPR), and include design elements that promote the development of aerobic granular sludge (AGS) with excellent settling properties, and may be incorporated into existing or new wastewater treatment systems. A WAS with polyphosphate accumulating organisms are transferred into a batch flow tank to release a soluble phosphorus under anaerobic conditions to promotes fermentation. The polyphosphate accumulating organisms within the WAS are transferred to a reactor vessel to release the soluble phosphorus and then to form a liquid supernatant and a settled WAS solids. The liquid supernatant is withdrawn and the settled WAS solids are retained within in the reactor vessel to ferment and form a liquid WAS phase over a settled WAS solids phase and release a soluble phosphorus component of the settled WAS solids into the liquid WAS phase, and then separate the liquid WAS phase enriched with the soluble phosphorus component from the settled WAS solids phase to create a phosphorus rich feedstock.

### TECHNICAL FIELD

The present disclosure relates to and is applicable to the processing of municipal and industrial wastewater in an activated sludge treatment process. It is particularly applicable to activated sludge treatment systems, which are configured to achieve “enhanced biological phosphorus removal” (EBPR), and include design elements that promote the development of “aerobic granular sludge” (AGS) with excellent settling properties, and may be applied to other activated sludge process configurations with similar benefits as described herein. The invention can be incorporated into existing or new “activated sludge wastewater” (ASW) treatment systems.

### BACKGROUND OF THE INVENTION

Biological nutrient removal (BNR) through improved methods employing “nitrification and denitrification” (NdN) and by “enhanced biological phosphorus removal” (EBPR) processes have gained widespread acceptance for the treatment of wastewater. These two biologically based methods and are generally preferable to physical-chemical treatment alternatives because of cost and sustainability considerations, and a lower carbon footprint with less greenhouse gas emissions due to reduced chemical use and lower energy needs.

An example of a conventional anaerobic, anoxic, and aerobic system for “nitrogen” (N) and “phosphorus” (P) removal is shown as prior art in FIG. 1, schematic process flow diagram of a typical “activated sludge wastewater treatment process” (ASWT) process 9 that employs an EBPR system 10 to utilize a “waste activated sludge” (WAS) 11, and where ammonia in an influent wastewater 12 is oxidized by autotrophic bacteria in an NdN process 13 to nitrate in an aerobic zone 14.

In the conventional ASWT process 9 with the basic EBRP system 10 as shown in FIG. 1, the process typically includes an anaerobic zone 15, followed by an anoxic zone 16, and then by one or more of the aerobic zone 14. More than one of each of these zones may be employed. This basic process design, is based upon the known “Modified Bardenpho Process,” with typically one or more anaerobic zones to promote EBPR, and also provides strong selective pressure against filamentous bacteria growth while at the same time promoting the growth of a denser, better settling biomass as the WAS 11.

A portion of settling biomass WAS 11 in the clarifier 18 may include an “aerobic granular sludge” (AGS) 17. There is a need to facilitate an efficient and effective “surface wasting” of the AGS included in WAS, to further help select against the growth of filamentous bacteria, but more importantly to help select for the denser constituents of the WAS, including the AGS.

The influent wastewater 12 is typically a municipal waste stream. A suspension of bacteria and other microorganisms referred to as a mixed liquor 13, which is received into and maintained in an aeration basin 15, for conversion into an “activated sludge” 20 that then is transferred to the clarifier 18. As shown in FIG. 1, the aeration basin effluent flows to the clarifier, where the mixed liquor settles and is returned to the aeration basin, and where the clarified effluent 19 as a treated wastewater overflows the clarifier for optional additional treatment, disinfection, and then disposal. A small fraction of the settled mixed liquor as WAS 11 is wasted in a clarifier WAS waste stream 25, typically to a digestion process, in order to maintain the desired concentration of mixed liquor in the aeration basin.

A large portion of the nitrate produced in the conventional ASWT process 9 with the basic EBRP system 10 is recycled to the upstream anoxic zone 16, where nitrate is used by denitrifying bacteria in the absence of oxygen to oxidize carbon provided in the influent wastewater 12. The low nitrate concentrations in the influent and a return activated sludge (RAS) 26 enables the first contact zone to be anaerobic, which provides an advantage for the selection of “polyphosphate accumulating organisms” (PAOs) 33. In the anaerobic contact zone the PAOs store “readily degradable carbon substrate” (rbCOD) in the influent wastewater in the form of “volatile fatty acids” (VFAs) as “polyhydroxyalkanoates” (PHA), by using the energy provided from glycogen resources and their release of stored phosphorus. In the aerobic zone and in the anoxic zone, PAOs oxidize the internally stored PHAs either with oxygen or with nitrate/nitrite, respectively. The energy provided by this oxidation results in phosphorus uptake and storage by PAOs, replenishment of their glycogen pool and production of more phosphorus-rich PAO biomass. Phosphorus is thus ultimately removed from the liquid phase due to storage in the PAOs that routinely leave the system with the “waste activated sludge” (WAS) 11.

The use of EBPR processes 10 instead of chemical precipitation for phosphorus removal facilitates phosphorus recovery. Under anaerobic conditions the PAOs 33 release phosphorus that can then be recovered for beneficial use as a fertilizer in the form of “magnesium ammonium phosphate” (MgNH₄PO₄×6H₂O), which is commonly referred to as a “struvite” 28.

Currently, struvite 28 production systems for use in “waste water treatment plants” (WWTPs) are available from several manufacturers. Such systems are currently being used in some larger size WWTPs in the United States, including Durham OR, Yakima WA, Boise ID, and York VA. These existing systems require anaerobic sludge digestion, with a typical process application depicted schematically in FIG. 2, also as prior art.

The WAS 11 from the EBPR system 10, and similarly by any conventional biological phosphorus removal process is thickened and subjected to anaerobic digestion where phosphorus is released by the PAOs. The “centrate” or filtrate from dewatering the digested sludge has a high phosphorus concentration, typically between approximately 300 mg/L and approximately 600 mg/L, which makes the filtrate suitable for struvite production in a phosphorus recovery process 27. Phosphorus recovery is considered an important part of all WWTPs to prevent the one-time use and wasting of this essential nutrient for food supply and all living organisms. However, only about 550 out of over 16,000 WWTPs in the United States employ anaerobic digestion, and most of these are the larger facilities. There are numerous smaller WWTPs for which anaerobic digestion is not cost-effective or feasible, so that the struvite recovery processes as shown in FIG. 2 are not presently possible.

There is a need for increasing the opportunity and availability for phosphorus recovery in WWTPs by addressing the needs of smaller size facilities. Types of WWTPs in need of improved phosphorus recovery processes and methods include “sequencing batch reactor” (SBR) processes, oxidation ditches and other small, activated sludge systems.

Smaller WWTPs have more often relied heavily on chemical precipitation, where P removal is required to protect effluent receiving waters. This is a very important consideration because P as a phosphate can be recovered as a byproduct of EBPR systems for beneficial use as fertilizer, while chemical precipitates of P typically are removed using metal salts and for all practical purposes are not recoverable. A key benefit of the present invention is to make possible a simple, low cost biological or a “soluble phosphorous” (SP) 30 recovery system for smaller WWTPs, with the improved technology and methods disclosed herein as can be applied to many plants without requiring anaerobic digestion.

Modern sanitation practice and large scale food production methods have broken the cycle of phosphorus (P) reuse in food production via the recycle of human waste in agriculture. Instead, there is a high dependence on inorganic phosphorus fertilizer to meet the nutrition needs of the world's growing population. At the same time, phosphate rock from which P fertilizers are produced is a non-renewable resource, which is being rapidly depleted. The collection and treatment of domestic wastewaters is also an essential part of modern life, but the discharge of phosphorus and nitrogen in effluent from “wastewater treatment plants” (WWTP's) contributes to the acceleration of eutrophication in surface waters and the deterioration of conditions for aquatic life. According to the USEPA, nearly every State has nutrient related pollution with negative impacts to over eighty significant estuaries and bays, and to thousands of rivers, streams, and lakes. More stringent discharge permit limits for effluent nutrients from WWTP's are becoming more frequent in efforts to minimize and prevent water quality impairment from eutrophication. A common effluent limit for phosphorus is a concentration of <0.10 mg/L, and for “nitrogen” (N) a “total nitrogen” (TN) concentration of <3.0 mg/L, and P is often the major nutrient of concern in freshwater impacted by WWTP's discharge or runoff.

### BRIEF DESCRIPTION OF THE DRAWINGS

Exemplary embodiments of the technology will become more fully apparent from the following descriptions and appended claims, taken in conjunction with the accompanying drawings. Understanding that these drawings depict only exemplary embodiments and are, therefore, not to be considered limiting of the scope of the technology, the exemplary embodiments will be described with additional specificity and detail through use of the accompanying drawings in which:

FIG. 1 is a schematic diagram of a conventional prior art biological nutrient removal process;

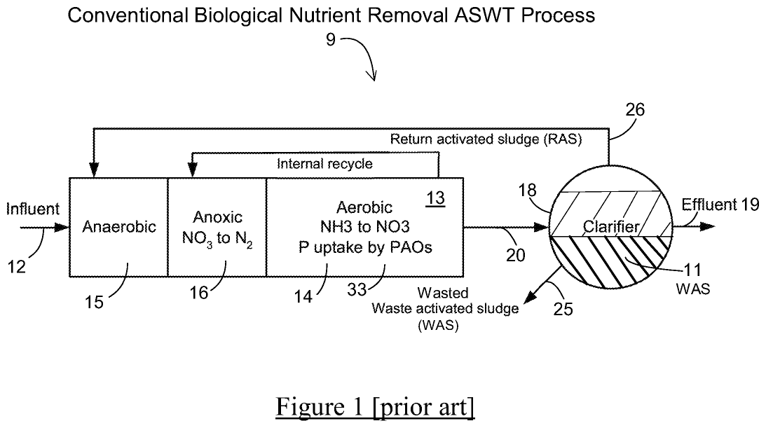

FIG. 2 is a schematic diagram of a conventional biological nutrient removal process with an enhanced biological phosphorus removal process and a phosphorous recovery as struvite.

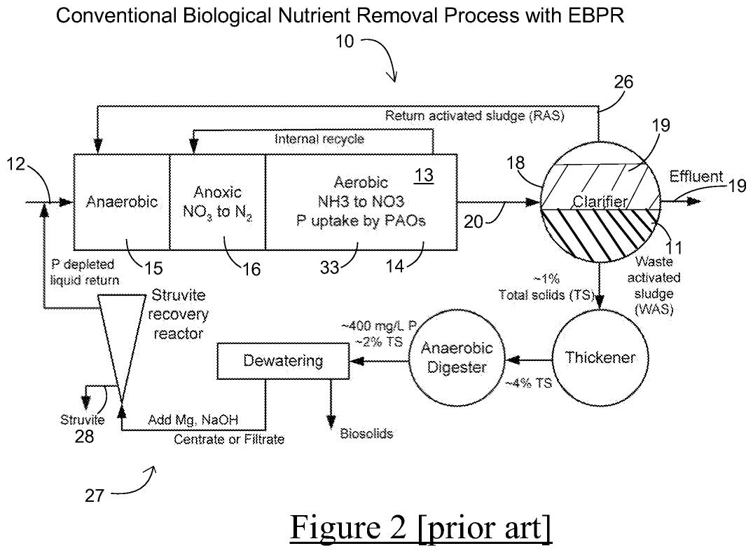

FIG. 3 is a schematic diagram of a biological nutrient removal process with phosphorous recovery as struvite, using the biological phosphorus recovery process and method of the present invention.

FIG. 4 is a schematic diagram of a biological nutrient removal process with phosphorous recovery as struvite for a direct land application, using the biological phosphorus recovery process and method of the present invention;

FIG. 5 is a chart showing phosphorus release under fermentation conditions of settled AGS waste activated sludge from a WWTP, located in Peshastin, WA;

FIG. 6 is a schematic diagram of a WAS fermentation, with a phosphorus release and recovery in a cyclic process reactor using the biological phosphorus recovery process and method of the present invention; and

FIG. 7 is a stepwise logic flowchart of an operational process embodiment of the phosphorus recovery method of the present invention.

Reference characters included in the above drawings indicate corresponding parts throughout the several views, as discussed herein. The description herein illustrates one preferred embodiment of the invention, in one form, and the description herein is not to be construed as limiting the scope of the invention in any manner. It should be understood that the above listed figures are not necessarily to scale and may include fragmentary views, graphic symbols, diagrammatic or schematic representations. Details that are not necessary for an understanding of the present invention by one skilled in the technology of the invention, or render other details difficult to perceive, may have been omitted.

### DETAILED DESCRIPTION OF SPECIFIC EMBODIMENTS

The present disclosure relates to and is applicable to the processing of municipal and industrial wastewater in an activated sludge treatment process. It is particularly applicable to activated sludge treatment systems, which are configured to achieve “enhanced biological phosphorus removal” (EBPR), and include design elements that promote the development of “aerobic granular sludge” (AGS) with excellent settling properties, and may be applied to other activated sludge process configurations with similar benefits as described herein. The invention can be incorporated into existing or new “activated sludge wastewater” (ASW) treatment systems.

The present invention pertains to a method for recovery of a biologically available phosphorus from an activated sludge wastewater treatment process. More specifically, the biological phosphorus recovery method results in phosphorus as a phosphate ion recovered in a soluble form, employing an anaerobic fermentation of a “waste activated sludge” (WAS) 11. A technical description of the present invention is provided herein by way of example. However, it should be noted that other similar configurations and components could be utilized in applying this method.

Exemplary embodiments of the “method for recovery of a biological phosphorus from an activated sludge wastewater treatment process” of the present invention, which more simply may be referred to herein as the “biological phosphorus recovery method” 100, will be best understood by reference to the drawings included herewith, with like parts designated by like numerals throughout. It will be readily understood that the components of apparatus elements employed in the biological phosphorus recovery method of the present invention, as generally described and illustrated in the figures herein, could be arranged and designed in a wide variety of different configurations. Thus, the following more detailed description of the embodiments of the apparatus, system, and method is not intended to limit the scope of the invention, as claimed, but is merely representative of exemplary embodiments of the technology.

The biological phosphorus recovery method 100 of the present invention can be applied to new and existing activated sludge process configurations to achieve improved performance in the basic activated sludge process, as well as “enhanced biological phosphorus removal” (EBPR) systems 10 and include design elements that promote the development of an “aerobic granular sludge” (AGS) 17, which has excellent settling properties and may be applied to other activated sludge process configurations with similar benefits as described herein.

The biological phosphorus recovery method 100 of the present invention can be incorporated into existing or new “activated sludge wastewater treatment” (ASWT) processes 9, to improve recovery of the phosphorus (P) as a soluble phosphorus (SP) 30 or biological phosphate, from the activated sludge and thereby improve operational energy efficiency of the system.

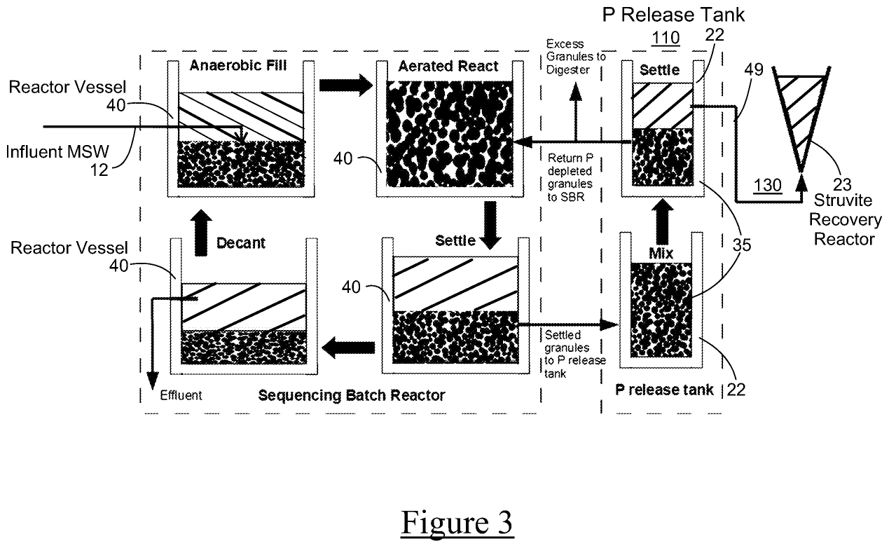

FIGS. 3 through 7, show features and steps of the biological phosphorus recovery method 100 of the present invention, as can be applied to new and existing treatment process designs as noted herein above, and again with note that other similar process configurations could be utilized in applying embodiments of the novel method steps disclosed and described herein.

The overall process of the biological phosphorus recovery method 100 includes combining a “sequencing batch reactor” (SBR) 21 containing a granular activated sludge 17 with a P release tank 22 and a struvite production reactor 23 for the production of the struvite 28, as depicted in FIG. 3. The phosphate concentrations in the main liquid process stream are low because the phosphate is released from the biomass in large flow volumes. Such high-volume flows would require very large and costly reactors for struvite recovery.

However, in the proposed approach for the biological phosphorus recovery method 100, a portion of the granular activated sludge 17 will be held in a separate tank in which the phosphate can be released into a smaller volume thus decreasing the liquid volume and increasing the phosphate concentrations. This will in turn decrease the size and cost of the struvite production reactor 23, and decrease usage of chemicals such as magnesium (Mg) and sodium hydroxide (NaOH). This advantage is realized because formation kinetics of struvite 28 are improved at higher phosphate concentrations.

Additionally, the recovered struvite 20 can be contaminated by sludge particles if the settling velocity of the sludge is poor. The use of a granular activate sludge 17 however, can avoid this problem due to its superior settling properties leading to a fast and reliable separation of water and sludge. This in turn results in a phosphate rich feedstock 90 in a liquid stream, which is mostly free of solids from which a high quality struvite 24 product can be recovered.

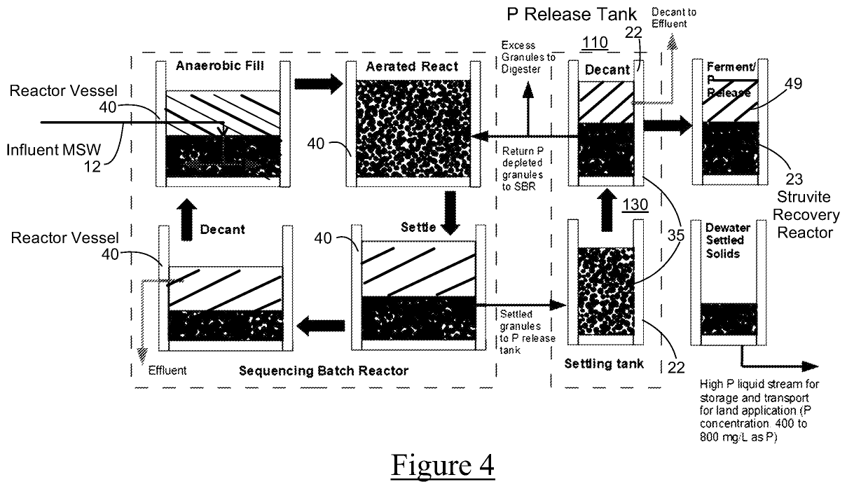

As an alternative to struvite recovery from the high P concentration liquid stream 90 from the P release tank, this liquid could be collected in a tank and then applied to agricultural land as fertilizer. This simple lost cost approach would be particularly applicable to small WWTPs in rural areas where farmland is located nearby. This alternative approach is depicted in FIG. 4 and is designed to maximize the concentration of P in the liquid stream which would be stored and subsequently applied to nearby land as fertilizer.

The recent developments in “granular activated sludge” (AGS) 17, will likely prove to be one of the most important advances in the “activated sludge wastewater treatment” (ASWT) process 9, in the approximately one hundred years since it was first developed. Important features of AGS for use with the present invention are its large biomass particle size, which typically ranges from between 0.5 mm to 2.0 mm, as shown in FIG. 4. Additionally, high density, rapid settling velocity, and function as a biofilm with different biological reactions within different layers of the AGS.

Particles of the AGS 17 consist of self-assembling microbial communities, which will form naturally in an activated sludge reactor when the right selective pressures are applied. Each granule is essentially a self-contained treatment system which simultaneously removes phosphorus, nitrogen, and biochemical oxygen demand (BOD) from the wastewater. The composition and structure of an aerobic granule is much more complex than the flocculent or “floc” formation of a conventional activated sludge reactor. In the granular sludge EBPR system, organic carbon is incorporated under anaerobic conditions into biomass as internal storage, polymer (PHA) thereby outcompeting aerobic fast growing heterotrophic bacteria for their substrate yielding smoother granules which settle well. The rapid settling of AGS makes possible a densely settled sludge blanket which when held under anaerobic conditions in a P release tank 22 can result in very high P concentrations in the liquid phosphate rich feedstock 90.

FIG. 3 is a diagram illustrating an EBPR Process 10 that processes an influent wastewater 12 as a “waste activated sludge” (WAS) 11 within an anaerobic zone 15. The ASWT) process 9 typically includes several “zones” of processing, including an “anoxic zone” 16 followed by an “aerobic zone” 14.

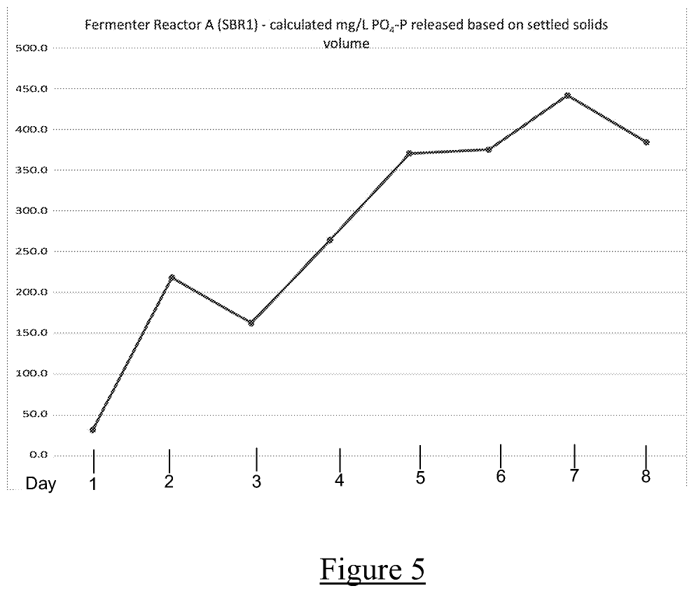

Fermentation of WAS 11 in the EBPR process 10 occurs within a reactor vessel 40, as shown in FIG. 4. Furthermore, FIG. 5 show a graph of typical results in an experiment designed to measure the rate of release of P over time when AGS 17 from an EBPR process is held under anaerobic or fermentation conditions. These experimental results demonstrate the efficacy of the biological phosphorus recovery method 100 of the present invention, in realizing the recovery of phosphorus (P) from municipal and industrial wastewater for beneficial use as fertilizer or other commercially important products that contain P.

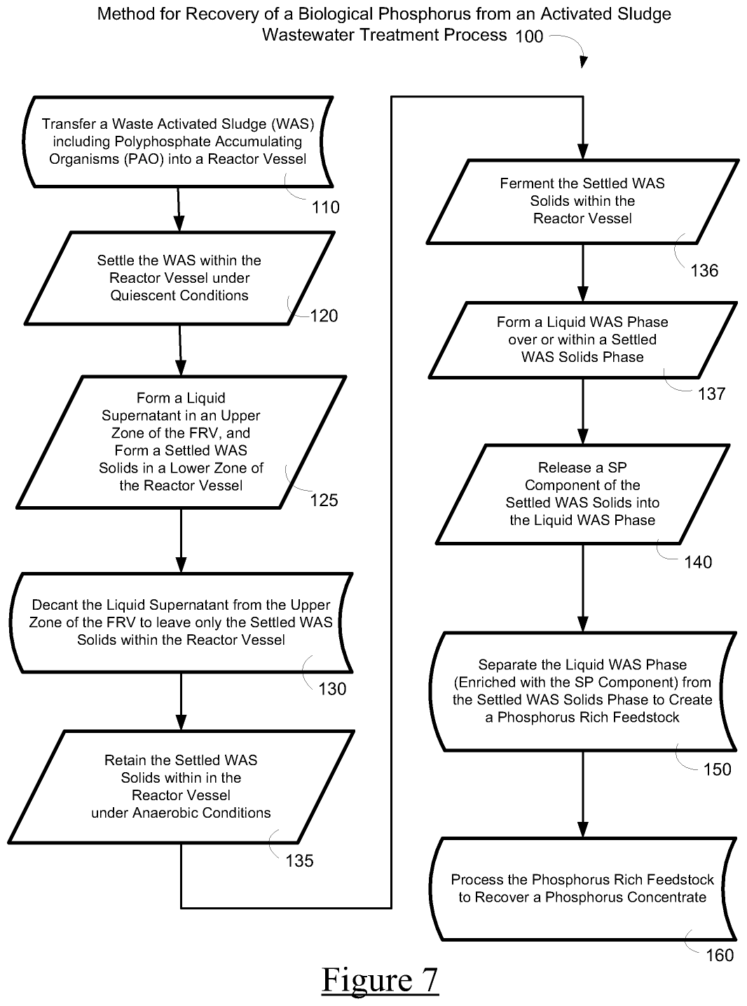

For the biological phosphorus recovery method of the present invention 100 vital process variables are input and monitored, most preferably to a microprocessor-based controller 50, enabling the accurate and precise execution and control of the process. These process variables include the process steps as shown in FIG. 7. The process variables input to the microprocessor-based controller, for use in a process control algorithm 51, as programmed into the microprocessor-based controller. The process control algorithm is most preferably a standard type of control algorithm written in a conventional programming code, to direct the logical output instructions of the microprocessor-based controller in the defined execution steps as disclosed herein. As a less preferred, alternative the process control algorithm can be executed from a personal or facility computer, or from a remote or a ‘cloud based’ processor or server.

The present invention can be incorporated into existing or new activated sludge wastewater treatment systems, to better achieve or improve “enhanced biological phosphorus removal” (EBPR), and “biological nitrogen removal” (BNR), by improving the settling of the activated sludge, along with increased energy efficiency. A preferred phosphorus recovery method 100 of the present invention as shown in FIGS. 3 and 4, incorporates the “fermentation reactor vessel” (FRV) 40, which together with an operation and control strategy maximizes the release of phosphate by polyphosphate accumulating organisms (PAOs) 33 under anaerobic conditions and then with fermentation process steps.

The biological phosphorus recovery method 100 of the present invention produces high concentrations of dissolved phosphorus as a phosphate (P) in aqueous solution derived from EBPR treatment systems 10 that process the “waste activated sludge” (WAS) 11, and especially from WAS treatment systems designed to promote the development of a settled WAS as the “aerobic granular sludge” AGS 35. Most preferably, the WAS containing the reactor vessel 40 is operated cyclically as described below, and as depicted in the process step diagram of FIG. 7, with first a transfer the WAS including the “polyphosphate accumulating organisms” (PAOs) 33 into the batch flow tank 31.

In the preferred process, the reactor vessel 40 is filled with the WAS 11 from an EBPR activated sludge system 10. The WAS settles in the reactor vessel to create a dense settled bed of thickened WAS as a “settled waste activated sludge solids (SWASS) 48 and a column of a liquid supernatant 49 as a substantially clear liquid above the settled bed of SWASS.

The liquid supernatant 49 is then decanted from the top of the reactor vessel 40 and the settled bed SWASS 48 is fermented aerobically to release the phosphate stored as a biological phosphorus (SP) 30 by the PAOs 33, as developed in the EBPR activated sludge process 10. Extract or separate the phosphate enriched liquid phase from the settled solids to create the phosphate rich feedstock 90 which can be further processed for beneficial reuse of recovered P.

The phosphorus recovery method 100 of the present invention recovers the biological or “soluble phosphorus” (SP) 30 as phosphate, from the activated sludge wastewater treatment process 9 utilizes a multiple of sequenced cycles to release the SP from the PAOs 33 present in the WAS 11 under anaerobic conditions to promote fermentation, within a settled sludge bed of the batch flow treatment process forms in the reactor vessel 40. The reactor vessel as typical batch flow treatment process includes a settled sludge bed, and the batch flow treatment process preferably utilizes a multiple of sequenced cycles so that the SP is released from the PAO under anaerobic conditions, which promotes fermentation within the settled sludge bed of the batch flow treatment process.

The ASWT 9 modified for use with the phosphorus recovery method 100 of the present invention includes the reactor vessel 40, and the PAO in the waste activated sludge (WAS) 11 from the enhanced biological phosphorus removal (EBPR) 10 wastewater treatment system is then transferred to the reactor vessel 110. Fermentation conditions are maintained in the reactor vessel to achieve the release of the soluble phosphorus from the WAS solids into the liquid phase of the WAS as a liquid supernatant 49 that is substantially clarified and typically clear.

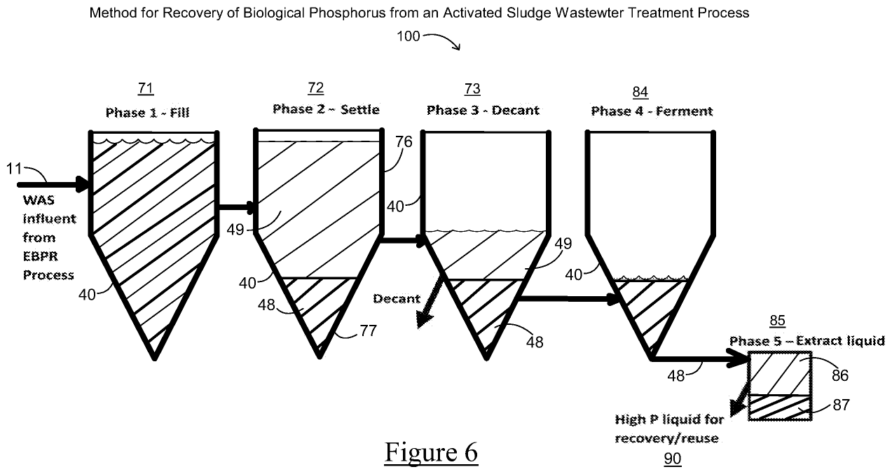

According to a most preferred embodiment of the phosphorus recovery method 100, as shown in FIG. 6, the sequential cycles within the reactor vessel 40 are controlled such that a batch process is created, essentially consisting of the following steps or cycles, beginning with a fill cycle 71, in which WAS is transferred into the reactor vessel. Then a settle cycle 72, in which the filling is terminated and the WAS contents of the reactor vessel are allowed to settle quickly within the reactor vessel, most preferably under substantially quiescent conditions 120.

As shown in FIG. 6, the reactor vessel 40 includes an upper zone 76, and a lower zone 77. A liquid supernatant 78 is formed in the upper zone, and the settled waste activated sludge solids (SWASS) 48 are formed in the lower zone of the reactor vessel 125. The liquid supernatant is substantially clear and free of suspended solids. Then in a decant cycle 73, is performed quickly, and most preferably within an hour of transfer into the reactor vessel, the liquid supernatant is decanted and withdrawn from the upper zone of the reactor vessel is withdrawn to leave only the SWASS within the reactor vessel 130.

The settled waste activated sludge solids (SWASS) 48 are retained within in the reactor vessel 40 under anaerobic conditions in a ferment cycle 84, to form a “liquid waste activated sludge” (LWAS) 86 containing phase over a “settled waste activated sludge solids” (SWASS) 87 containing phase. The ferment cycle achieves the release of the soluble phosphorus (SP) 30 component of the SWASS into the LWAS phase as a result of fermentation within the settled bed of the FRV. Specifically for the ferment cycle, the SWASS are retained within in the reactor vessel under anaerobic conditions 135, and the settled waste activated sludge solids are then fermented within the reactor vessel 136 to form the LWAS phase over or within the SWASS phase 137. Typically, the LWAS phase is comingled with the SWASS phase and may require a mechanical separation step to better separate the two phases, but these ferment cycle 84 process steps serve to produce the desired release of the SP component of the SWASS into the LWAS phase 140.

The ferment cycle 84 is most preferably followed by an extract cycle 85, in which the SP 30 enriched LWAS 86 phase as enriched with the SP component is separated from the SWASS 48 phase. This extraction creates the phosphorus rich feedstock 90 within the reactor vessel 40. Specifically, the soluble phosphorus enriched liquid phase is separated from the liquid LWAS, from the SWASS to create the phosphorus rich feedstock 150.

At the end of the ferment cycle 84 as defined above, the liquid phase of the settled bed of fermented solids will be extracted utilizing a form of mechanical thickening or dewatering, with such methods well-known by those skilled in processing technology for ASWT processes 9. The liquid phase, which is substantially clear will have a high concentration of SP 30 can then be further processed in a downstream reactor producing struvite or applied directly to agricultural land as a fertilizer. FIG. 6 is a schematic depicting the above discussed cycles, implemented sequentially in the reactor vessel 40.

This is a beneficial reuse of the phosphorus rich feedstock 90, which can be processed by conventional methods of phosphate solution concentration to recover a phosphorus concentrate 91. With the phosphorus rich feedstock processed to recover a phosphorus concentrate 160, a highly beneficial and commercially desirable fertilizer adjuvant is obtained.

Again, the phosphorus recovery method 100 of the present invention can be applied to new and existing activated sludge process configurations to achieve an improved EBPR system 10, and especially in those existing systems designed and operated to support the development of AGS 17. The above technical description of the innovations of the present invention is by way of example, however, it should be noted that other similar process configurations could be utilized in applying this method. For instance, FIGS. 3 and 4 show process schematics of preferred embodiments of the present invention which provides a method for simple, low-cost recovery of phosphorus particularly at small municipal and industrial waste water treatment plants.

An objective of the phosphorus recovery method 100 of the present invention is to provide in the ferment cycle 84, an anaerobic fermentation period in the reactor vessel 40, into which WAS 11 from the EBPR 10 process has been introduced and held for a period of time which will maximize the release of SP 30 to the LWAS 86 as the phosphorus rich feedstock 90.

Another objective of the present invention includes employing an automated a microprocessor-based controller 50, enabling the accurate and precise execution and control of the process. The process variables input to the microprocessor-based controller, for use in a process control algorithm 51 system preferably include the telemetered measurement of oxidation-reduction potential (ORP), liquid level sensors, and liquid solids interface sensors as process variable inputs to automate the fill, settle, decant, and ferment cycles to optimize phosphorus release into the liquid phase.

In the preferred embodiment of the present invention, anaerobic conditions and fermentation will occur in the settled bed of waste EBPR sludge where anaerobic conditions are indicated when “oxidation reduction potential values” ORP of −300 mV or less, to optimize release of the phosphorus, with the optimum duration for the ferment cycle defined as a point in time when a second derivative of the ORP versus time reaches a preferred value. For instance, the duration of the ferment cycle 84 will be at least approximately twenty-four hours but may be as long as up to four or five days, in order to optimize phosphorus release. The optimum duration for the ferment cycle can be defined a point in time when the second derivative of the ORP vs. time reaches the most preferred, predetermined setpoint of approximately 2.0.

For this Detailed Description of Specific Embodiments, the terms “connected”, “attached”, “coupled” and “mounted” refer to any form of interaction between two or more elements, including mechanical, electrical, magnetic, electromagnetic, fluid, and thermal interaction. Two components may be functionally coupled with or to each other, even though they are not in direct contact with each other.

Also, the terms “substantially”, and “approximately” or “approximate” are employed herein throughout, including this detailed description and the attached claims, with the understanding that is denotes a level of exactitude commensurate with the skill and precision typical for the particular field of endeavor, as applicable.

Additionally, the terminology used in this Detailed Description of Specific Embodiments is to be interpreted according to ordinary and customary usage in the field of the invention as exemplified in the pertinent U.S. and International Patent Classification Codes, and equivalent codes in other patent classification systems.

The word “embodiment” is used herein to mean “serving as an example, instance, or illustration.” Any embodiment described herein is not necessarily to be construed as preferred or advantageous over other embodiments. While the various aspects of the embodiments are presented in drawings, the drawings are not necessarily drawn to scale.

Additionally, reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that the above Detailed Description of Specific Embodiments includes the referenced figures and following claims, and is more simply referred to herein as the “description” or the “disclosure.” In this description, various features are sometimes grouped together in a single embodiment, figure, or written explanation thereof for the purpose of streamlining this disclosure. However, this method of disclosure is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this description are hereby expressly incorporated into this description and disclosure, with each claim standing on its own as a separate embodiment. This description includes all permutations of the independent claims with their dependent claims.

In compliance with the statutes, the invention has been described in language more or less specific as to structural features and process steps where applicable. While this invention is susceptible to embodiment in different forms, the specification illustrates preferred embodiments of the invention with the understanding that the present disclosure is to be considered an exemplification of the principles of the invention, and the disclosure is not intended to limit the invention to the particular embodiments described. Those with ordinary skill in the art will appreciate that other embodiments and variations of the invention are possible, which employ the same inventive concepts as described above. Therefore, the invention is not to be limited except by the following claims, as appropriately interpreted in accordance with the doctrine of equivalents.

What is claimed is:

1. A method for recovery of a biological phosphorus from an activated sludge wastewater treatment process, said method comprising the steps of:
a) transferring a waste activated sludge into a reactor vessel, the waste activated sludge including a polyphosphate accumulating organism;
b) settling the waste activated sludge within the reactor vessel under quiescent conditions;
c) forming a liquid supernatant in an upper zone of the reactor vessel, and forming a settled waste activated sludge solids in a lower zone of the reactor vessel;
d) decanting the liquid supernatant from the upper zone of the reactor vessel;
e) retaining the settled waste activated sludge solids within in the reactor vessel under anaerobic conditions;
f) fermenting the settled waste activated sludge solids within the reactor vessel to form a liquid waste activated sludge phase and a settled waste activated sludge solids phase;
g) releasing a soluble phosphorus component of the settled waste activated sludge solids into the liquid activated sludge phase; and
h) separating the liquid activated sludge phase enriched with the soluble phosphorus component from the settled activated sludge solids phase, to create a phosphorus rich feedstock.

2. The method of claim 1, additionally including the step of:
i) processing the phosphorus rich feedstock to recover a phosphorus concentrate.

3. The method of claim 1, wherein the step of decanting the liquid supernatant from the upper zone of the reactor vessel, additionally requires that said decanting is performed before a release of the soluble phosphorus component into the liquid supernatant.

4. The method of claim 1, wherein the step of fermenting the settled waste activated sludge solids within the reactor vessel to form a liquid waste activated sludge phase and a settled waste activated sludge solids phase additionally includes monitoring and maintaining anaerobic conditions with an oxidation reduction potential values of −300 mV or less, to optimize release of the phosphorus, with the optimum duration for the ferment cycle defined as a point in time when a second derivative of the oxidation reduction potential value versus time reaches a value of approximately 2.0.

5. The method of claim 1, wherein a microprocessor-based monitoring and control system is utilized to control and optimize the biological phosphorus from an activated sludge wastewater treatment process.

6. The method of claim 5, wherein an automated analyzer measuring soluble phosphorus in the liquid phase is used as an input to the microprocessor based monitoring and control system to control the duration of the ferment cycle.
